(19) United States
(12) Patent Application Publication
(10) Pub. No.: US 2024/0123152 A1
(43) Pub. Date: Apr. 18, 2024

(54) ERGONOMIC SYRINGE ADAPTOR

(71) Applicant: Bobby Nourani, Long Beach, CA (US)

(72) Inventors: Bobby Nourani, Long Beach, CA (US); Michael Stephen Rafferty, Madison, WI (US); Hooshmand Nourani, Long Beach, CA (US)

(73) Assignee: Bobby Nourani, Long Beach, CA (US)

(21) Appl. No.: 18/277,664

(22) PCT Filed: Feb. 17, 2022
(86) PCT No.: PCT/US22/16814

§ 371 Date: Aug. 17, 2023

Related U.S. Application Data
(60) Provisional application No. 63/150,734, filed on Feb. 18, 2021.

(51) Int. Cl. A61M 5/31

(52) U.S. Cl. CPC A61M 5/3137; A61M 2207/10

(57) ABSTRACT

Syringe adaptors comprise a hub portion comprising an outer surface, an inner surface, a top surface and a bottom surface, the inner surface configured to receive a portion of a syringe body; and a grip portion. The inner surface may comprise at least one projection.

### CROSS-REFERENCE TO RELATED APPLICATIONS

This application claims the benefit of U.S. Provisional Application No. 63/150,734 filed Feb. 18, 2021, the contents of which are incorporated by reference herein.

### FIELD OF THE DISCLOSURE

The present disclosure is generally directed to medical devices and in particular syringes which can be used to deliver medications as well as to inject and to extract fluids. More specifically, the present disclosure relates to handles and adaptors for syringes.

### INTRODUCTION

Since their beginnings in 1961 by the medical supply company Becton Dickinson, syringes have transformed the way medical professionals are able to deliver drugs, inject medications, and extract fluids. A typical syringe includes a syringe body and a plunger movably positioned within the syringe body. The syringe body generally includes a barrel portion where the fluid to be injected is contained, an open end configured to receive the plunger and a flange. Some syringes comprise a necked end configured to receive a needle.

### SUMMARY

The present disclosure generally relates to syringes, syringe handles, syringe adaptors and modifications thereof. Syringe “handle” and syringe “adaptor” may be used interchangeably throughout this disclosure. Advantageously, and without being bound by any particular theory, exemplary embodiments set forth in this disclosure present various designs that distribute the force required from an operator used to operate a syringe thereby reducing fatigue and offering a more comfortable use.

In general, a syringe handle includes a hub portion and a grip portion. The hub portion comprises an inner surface configured to receive the body of a syringe, an outside surface, a top surface and a bottom surface. The grip portion includes at least one arm. In an exemplary embodiment, the grip portion includes a first and a second arm which are connected to and extend from the outside surface of the hub portion. In an exemplary embodiment, the first arm is biased toward the top surface of the hub portion and the second arm is biased toward the bottom surface of the hub portion.

A syringe may be modified with or comprise an integral grip portion. In an exemplary embodiment, a syringe user inserts the body of a syringe into the channel defined by the inner surface of the hub portion of the syringe handle. The handle is inserted such that either the top surface or the bottom surface of the hub abuts the flange of the syringe. The hub may be placed onto the syringe barrel in an axial direction. Alternatively, the hub may be placed radially (e.g. snaped-on) onto the syringe barrel. In typical operation, the user depresses the plunger of the syringe by placing their thumb or palm on the plunger and a number of fingers on the syringe arms.

The orientation of the arms on the syringe handle positions a user's hand in an ergonomically advantageous position during use of the syringe. In addition, the arms provide a greater surface area for the user to place their fingers while depressing the plunger of a syringe. By facilitating an ergonomically advantageous position and increasing the surface area, the user is better able to control the syringe during injections. In addition, and without being bound by any theory, the increased surface area is believed to allow the user to apply more force to the plunger and reduce the relative pressure on the fingers/hand/wrist of the user which would otherwise be in contact with the flange of the syringe. This is believed to result results in the user experiencing less fatigue/strain when performing multiple injections, as is done, for example, in prolotherapy.

The details of one or more embodiments are set forth in the accompanying drawings and the description below. Other features, objects, and advantages of these embodiments will be apparent from the description, drawings, and claims. Moreover, it is to be understood that both the foregoing summary of the disclosure and the following detailed description are exemplary and intended to provide further explanation without limiting the scope of the disclosure.

According to an exemplary embodiment, a syringe adaptor comprises a grip portion, a hub portion comprising an inner surface and an outer surface, a channel defined by the inner surface, a central longitudinal axis passing through the channel, and at least one projection extending away from the inner surface. The at least one projection may be configured to engage a syringe barrel received in the channel. The grip portion may extend from the hub outer surface away from the central axis. In an exemplary embodiment, the grip portion comprises a first arm and a second arm.

### BRIEF DESCRIPTION OF THE DRAWINGS

FIG. 1 is a front view of a syringe with a syringe handle.

FIG. 2 is a front perspective view of a syringe handle.

FIG. 3 is a front view of the syringe handle shown in FIG. 2.

FIG. 4 is a side view of the syringe handle shown in FIG. 2.

FIG. 5 is a bottom view of the syringe handle shown in FIG. 2.

FIG. 6 is a front perspective view of another example embodiment of a syringe handle.

FIG. 7 is a front view of the syringe handle shown in FIG. 6.

FIG. 8 is a side view of the syringe handle shown in FIG. 6.

FIG. 9 is a bottom view of the syringe handle shown in FIG. 6.

FIG. 10 is a front perspective view of another example embodiment of a syringe handle.

FIG. 11 is a top cross-sectional view of a syringe adaptor according to an exemplary embodiment.

FIG. 12 is a top cross-sectional view of a syringe adaptor according to an exemplary embodiment.

FIG. 13 is a top cross-sectional view of a syringe adaptor according to an exemplary embodiment.

FIG. 14 is a top cross-sectional view of a syringe adaptor according to an exemplary embodiment.

FIG. 15 is a top cross-sectional view of a syringe adaptor according to an exemplary embodiment.

FIG. 16 is a top cross-sectional view of a syringe adaptor according to an exemplary embodiment.

FIG. 17a-f are side cross-sectional views of projection shapes according to several exemplary embodiments.

FIG. 18 is top cross-sectional view of a syringe adaptor according to an exemplary embodiment.

FIG. 19 is a top cross-sectional of a syringe adaptor according to an exemplary embodiment.

FIG. 20 is a top cross-sectional of a syringe adaptor according to an exemplary embodiment.

FIG. 21 is a top cross-sectional of a syringe adaptor according to an exemplary embodiment.

FIG. 22 is a top cross-sectional of a syringe adaptor according to an exemplary embodiment.

FIG. 23 is a top cross-sectional of a syringe adaptor according to an exemplary embodiment.

FIG. 24 is a perspective view of a syringe adaptor according to an exemplary embodiment.

FIG. 25 is a side view of a syringe adaptor according to an exemplary embodiment.

FIG. 26 is a side view of a syringe adaptor according to an exemplary embodiment.

FIG. 27 is a side view of a syringe adaptor according to an exemplary embodiment.

FIG. 28 is a side view of a syringe adaptor according to an exemplary embodiment.

FIG. 29 is a top view of a syringe adaptor according to an exemplary embodiment.

FIG. 30 is a bottom view of a syringe adaptor according to an exemplary embodiment.

FIG. 31 is a partial cross-sectional view along line 8-8 of FIG. 29.

FIG. 32 is a perspective view a syringe adaptor according to an exemplary embodiment.

FIG. 33 is a side view a syringe adaptor holding a syringe barrel according to an exemplary embodiment.

FIG. 34 is a perspective view a syringe adaptor according to an exemplary embodiment.

FIG. 35 is a perspective view a syringe adaptor according to an exemplary embodiment.

FIG. 36 is a perspective view a syringe adaptor according to an exemplary embodiment.

FIG. 37 is a perspective view a syringe adaptor according to an exemplary embodiment.

FIG. 38 is a perspective view a syringe adaptor according to an exemplary embodiment.

FIG. 39 is a perspective view a syringe adaptor according to an exemplary embodiment.

FIG. 40 is a perspective view a syringe adaptor according to an exemplary embodiment.

FIG. 41 is a perspective view a syringe adaptor according to an exemplary embodiment.

FIG. 42 is a top view a syringe adaptor according to an exemplary embodiment.

FIG. 43 is a bottom view a syringe adaptor according to an exemplary embodiment.

FIG. 44 is a side view a syringe adaptor according to an exemplary embodiment.

FIG. 45 is a side view a syringe adaptor according to an exemplary embodiment.

FIG. 46 is a side view a syringe adaptor according to an exemplary embodiment.

FIG. 47 is a side view a syringe adaptor according to an exemplary embodiment.

FIG. 48 is a perspective view of a syringe adaptor according to an exemplary embodiment.

FIG. 49 is a front elevational view of a syringe adaptor according to an exemplary embodiment.

FIG. 50 is a rear elevational view of a syringe adaptor according to an exemplary embodiment.

FIG. 51 is a left side elevational view of a syringe adaptor according to an exemplary embodiment.

FIG. 52 is a right side elevational view of a syringe adaptor according to an exemplary embodiment.

FIG. 53 is a top plan view of a syringe adaptor according to an exemplary embodiment.

FIG. 54 is a bottom plan view of a syringe adaptor according to an exemplary embodiment.

FIG. 55 is a partial cross-sectional view of the syringe adaptor taken along line 28-28 of FIG. 53.

FIG. 56 is a lower perspective view of a syringe adaptor according to an exemplary embodiment.

FIG. 57 is further front elevational view of a syringe adaptor according to an exemplary embodiment, where in the syringe holder is shown holding a syringe barrel.

FIG. 58 is an upper perspective view of a syringe adaptor according to an exemplary embodiment.

FIG. 59 is a front elevational view of a syringe adaptor according to an exemplary embodiment.

FIG. 60 is a rear elevational view of a syringe adaptor according to an exemplary embodiment.

FIG. 61 is a left side elevational view of a syringe adaptor according to an exemplary embodiment.

FIG. 62 is a right side elevational view of a syringe adaptor according to an exemplary embodiment.

FIG. 63 is a top plan view of a syringe adaptor according to an exemplary embodiment.

FIG. 64 is a bottom plan view of a syringe adaptor according to an exemplary embodiment.

FIG. 65 is a partial cross-sectional view of the syringe adaptor taken along line 38-38 of FIG. 63.

FIG. 66 is a lower perspective view of a syringe adaptor according to an exemplary embodiment.

FIG. 67 is further front elevational view of a syringe adaptor according to an exemplary embodiment, where in the syringe holder is shown holding a syringe barrel.

FIG. 68 is a side cutaway view of a syringe adaptor according to an exemplary embodiment.

FIG. 69 is an elevational cutaway view of a syringe adaptor according to an exemplary embodiment.

FIG. 70 is a perspective view of a syringe adaptor according to an exemplary embodiment.

FIG. 71 is a side cutaway view of a syringe adaptor according to an exemplary embodiment.

FIG. 72 is a side cutaway view of a syringe adaptor according to an exemplary embodiment.

### DETAILED DESCRIPTION OF THE EMBODIMENTS

Various embodiments of the present disclosure will be described in detail with reference to the drawings, wherein like reference numerals represent like parts and assemblies throughout the several views. The features described herein are examples of implementations of certain inventive aspects which underlie the disclosure.

As briefly described above, embodiments of the present disclosure are directed to syringe handles designed to provide an ergonomic grip. Syringe handles are described below as used with example syringes. In some instances, syringe handles disclosed and contemplated herein can be retrofit onto previously-manufactured syringes. In some instances, syringe handles disclosed and contemplated herein can be integral to a syringe body.

The syringe handle and its various described embodiments may be constructed from a variety of materials. In some instances, it may be advantageous for the syringe handle to be made from metal or another sterilizable material to permit the syringe handle to be reused. In other instances, it may be advantageous for the syringe handle to be made of plastics, specifically medical grade plastics, resin, rubber, fiberglass, or any combination thereof. The syringe handles described above may be made from a single material or any combination thereof.

Syringe use spans a variety of medical, biotech, and related fields, including prolotherapy, cosmetic industry (e.g. dermal fillers, botox, etc.) vaccination administration, blood transfusions, self-administered injections (e.g. diabetes, fertility, etc.) and laboratory bench science technicians, as well as both dental and veterinary applications. Typical operations of the syringe can cause discomfort over prolonged and repetitive use, indicating that the benefits from a more ergonomically advantageous syringe would be beneficial to a large variety of professionals.

Typical use of a syringe involves the user placing their thumb (or sometimes palm) on the plunger of the syringe and placing two fingers on the flange of the syringe. The user then depresses the plunger of the syringe by applying force to both the plunger and the flange of the syringe. This results in the plunger being pushed into the barrel of the syringe which reduces the interior volume and forces the contained fluid through the necked end. The amount of force required to depress the syringe varies depending on the surface area of the plunger, volume of the syringe and the diameter of the needle attached to the necked end. It may also depend on the composition of the fluid being administered. Of course, use of syringes without needles is also improved with the present designs. For instance, the present disclosure may apply to certain dental syringes that do not accept a needle. Such devices are tapered to allow for expression of high-pressured fluid (i.e., monoject). In yet another example, the present disclosure is applicable to nurses or other technicians may use a syringe without a needle on an IV and express medication.

Physicians who utilize syringes on a frequent basis, particularly those performing injections, find that they experience discomfort, sometimes significantly, specifically in the hand, wrist and thumb (sometimes referred to as a repetitive strain injury). The discomfort may also appear in the upper extremity including the shoulder from the repetitive strain. The pain experienced by these physicians generally results from a high volume of fluid being ejected with repetitive use and increased resistance from the syringe end and/or the sub-millimeter-wide needles. By reducing and potentially eliminating the pain experienced while using a syringe, physicians can reduce the number of workdays lost due to injury, and aging doctors, surgeons, laboratory technicians, dentists, and veterinarians can remain active longer into their careers. Moreover, the practice of the individual may improve since the increased contact surface area allows for better control of syringe.

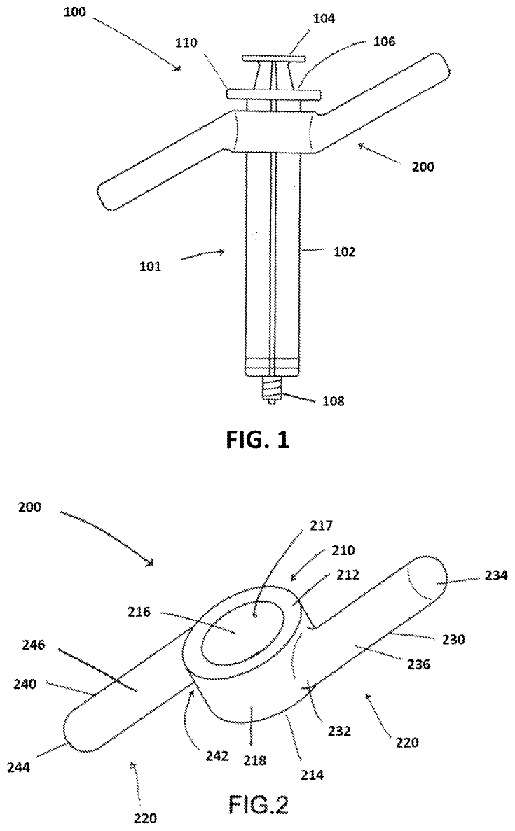

FIG. 1 illustrates an example syringe 100. Syringe 100 generally includes a body portion 101 and a plunger 104. Body portion 101 includes a barrel portion 102 that receives a plunger 104 at open end 106 and a necked end 108, which can be configured to receive a needle. Open end 106 is surrounded by a flange 110 to allow for the user to grip the syringe 100 while depressing the plunger 104. Example syringe handle 200 is described in greater detail below with reference to FIGS. 2-5.

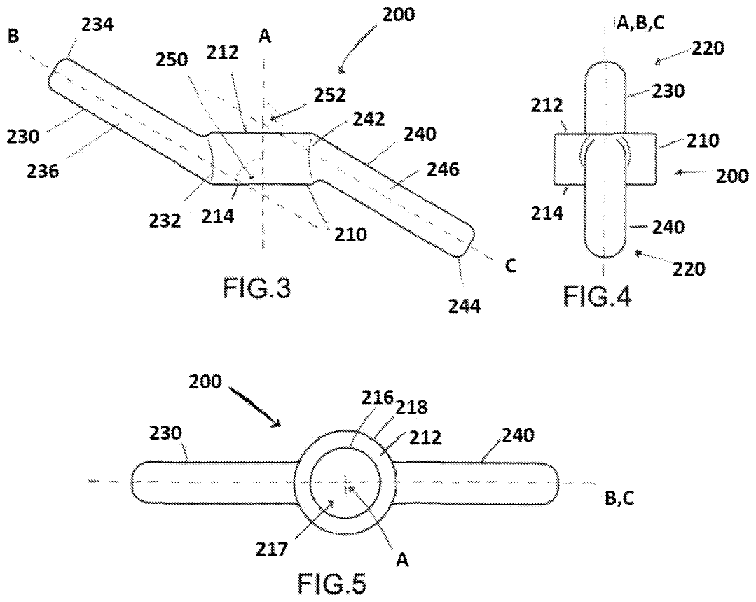

FIG. 2 illustrates a top perspective view of an example syringe handle 200. FIG. 3 illustrates a front view of syringe handle 200. FIG. 4 illustrates a side view of syringe handle 200. FIG. 5 illustrates a top view of syringe handle 200.

Syringe handle 200 includes a hub portion 210 and a grip portion 220. The hub portion 210 includes a top surface 212, a bottom surface 214, an inner surface 216 and an outer surface 218. Both inner surface 216 and outer surface 218 are disposed between top surface 212 and bottom surface 214.

Inner surface 216 forms a channel 217 in the hub portion 210 configured to receive a syringe barrel 102. Inner surface 216 is generally curved and configured to receive syringe barrel 102 and includes an axis A. Axis A is positioned along the length of the channel 217 defined by inner surface 216 and passes through the center of gravity of the channel 217. In some embodiments, inner surface 216 is cylindrical and axis A is a central axis passing through the channel 217 defined by inner surface 216. In other embodiments, inner surface 216 may be configured to accommodate different syringe barrel 102 shapes. The channel 217 formed by inner surface 216 can be variously sized depending upon the application, which enables syringe handle 202 to be fitted to different brands and/or syringe sizes.

Top surface 212 and bottom surface 214 are configured such they can provide a seat for the flange 110 of a syringe 100. When syringe handle 200 is positioned for operation, either the top surface 212 or the bottom surface 214 abuts flange 110 of said syringe 100.

The grip portion 220 includes a first arm 230 and a second arm 240. The first arm 230 includes a connection point 232 opposite a distal end 234, an outer surface 236, and an axis B. The second arm 240 includes a connection point 242 opposite a distal end 244, an outer surface 246, and an axis C.

The first arm 230 is connected to the outer surface 218 of the hub portion 210 at connection point 232. The first arm 230 extends from outer surface 218 at connection point 232 and terminates at distal end 234. Distal end 234 is biased toward the top surface 212 of the hub portion 210. The second arm 240 is connected to outer surface 218, opposite the first arm 230, at connection point 242. In some embodiments, connection point 232 and connection point 242 are positioned on opposite sides of hub portion 210. The second arm 240 extends from outer surface 218 at connection point 242 and terminates at distal end 244. Distal end 244 is biased toward the bottom surface 214 of the hub portion 210.

Outer surface 236 and outer surface 246 may be of constant cross-section or contoured to facilitate the placement of fingers. Additionally, outer surface 236 and outer surface 246 may be textured by knurling or otherwise to improve grip, or may be covered in a material, such as rubber or soft plastic, that provides for increased grip by the user.

In various embodiments, the first arm 230, the second arm 240, or both may have various cross-sectional shapes along at least a portion of the arm including the entire length of the arm. For instance, in an exemplary embodiment, the first arm 230, the second arm 240 or both comprise an elliptical cross-section. In another exemplary embodiment, the first arm 230, the second arm 240, or both comprise a rectangular cross-section having rounded corners. In an exemplary embodiment, first arm 230 and second arm 240 are cylindrical bodies including circular cylindrical and elliptical cylindrical shapes. In at least one variant of such embodiments, axis B is the central longitudinal axis of the first arm 230 and axis C is the central longitudinal axis of the second arm 240 such that the axes of the first arm and second arm extend radially in planar alignment at 180 degrees from one another, along the central axis of the open-ended split-hub portion. In one variant of the exemplary embodiments, the first arm 230 and the second arm 240 may have cross sections that are non-circular, such that outer surface 246 in which case axis B is oriented along the length of the first arm 230 and passes through the center of gravity of the first arm 230 and axis C is oriented along the length of the second arm 240 and passes through the center of gravity of the second arm 240. In an exemplary embodiment, the first arm 230, the second arm 240 or both may have a continuous, smooth and uninterrupted exterior surface along at least a portion of each arm, including the entire length of the arm. In at least one exemplary embodiment at least a portion of the exterior surface of the first arm 230, the second arm 240, or both comprises a textured portion to advantageously enhance a user's ability to hold and operate the device without added strain.

The first arm 230 of the grip portion 220 extends radially from the outer surface 218 of the hub portion 210 such that the distal end 234 is biased with respect to of axis A and toward the top surface 212 of the hub portion 210, forming angle 250 between axis A and axis B. In various embodiments, the angle 250 is greater than 0 degrees and less than 90 degrees. In some embodiments, the angle 250 formed between axis A and axis B is from 20 to 65 degrees. In some embodiments, the angle 250 is from 30 to 50 degrees. In some embodiments, the angle 250 is from 30 to 40 degrees. In some embodiments, the angle 250 is from 55 to 65 degrees. In an exemplary embodiment, the first arm 230, the second arm 240 or both extend radially from a connection point on the hub portion 210. Accordingly, the cross-sectional area of the first arm 230, the second arm 240, or both, at the connection point may be located entirely between the top surface and bottom surface of the hub portion.

The second arm 240 of the grip portion 220 extends radially from the outer surface 218 of the hub portion 210 such that the distal end 244 is biased along the direction of axis A and toward the top surface 212 of the hub portion 210, forming angle 252 between axis A and axis C. In various embodiments, the angle 252 is greater than 90 degrees and less than 180 degrees. In some embodiments, the angle 252 formed between axis A and axis C is from 110 to 150 degrees. In some embodiments, the angle 252 formed between axis A and axis C is from 120 to 40 degrees. In some embodiments, the angle 252 formed between axis A and axis C is from 135 to 145 degrees. In some embodiments, angle 250 is the supplement to angle 252 such that the sum of angle 250 and angle 252 is 180 degrees.

In some embodiments, the first arm 230 and the second arm 240 are configured to be parallel such that axis B and axis C are parallel to each other. In other embodiments, the first arm 230 and the second arm 240 may be configured in parallel such that axis B and axis C are coincident.

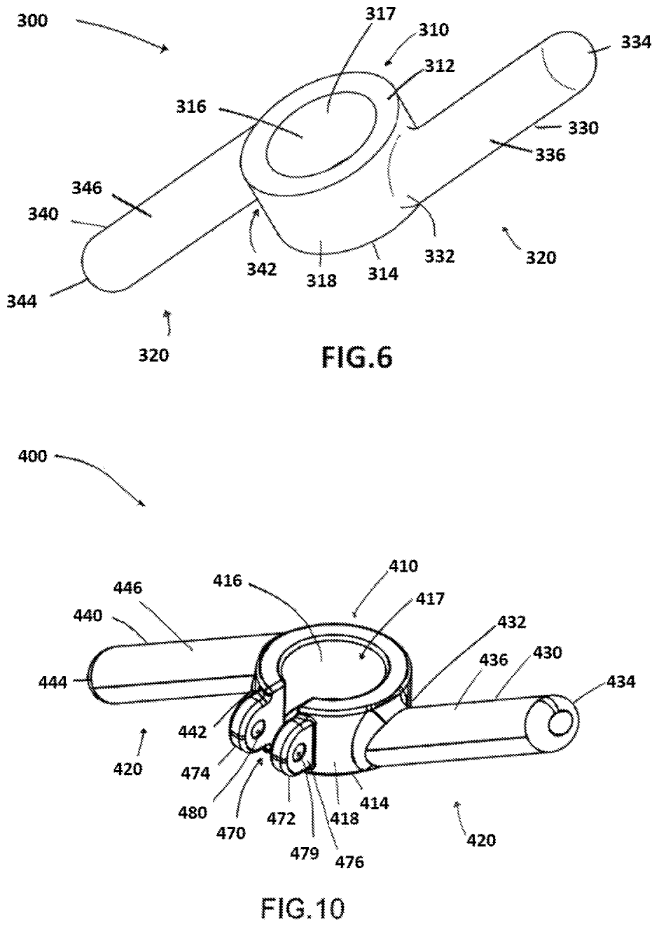

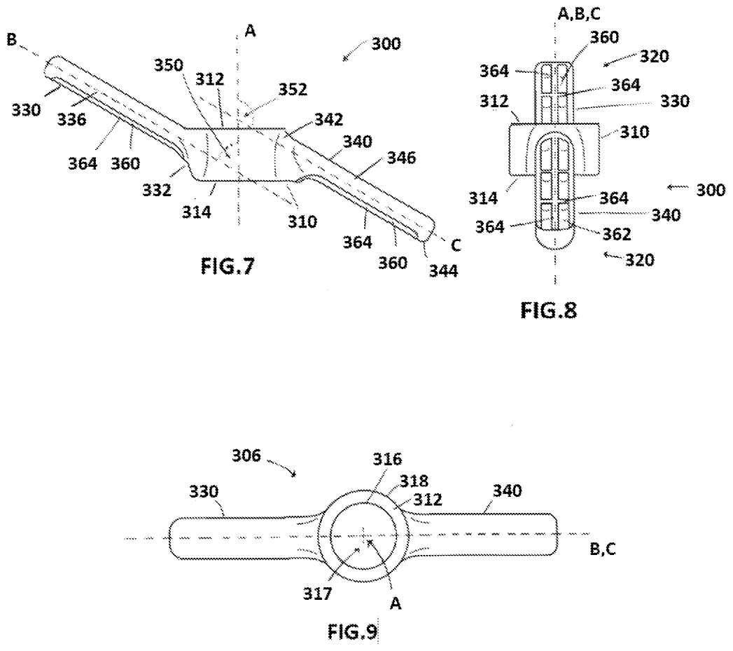

FIGS. 6-9 illustrate another embodiment of a syringe handle 300. Syringe handle 300 is similar to the assembly shown in FIGS. 2-5 and common elements have similar reference numerals as the embodiment shown in FIGS. 2-5.

The outer surface 336 of the first arm 330 and the outer surface 346 of the second arm 340 have a U-shaped cross section defining cavity 360 in the first arm 330 and cavity 362 in the second arm. Both cavity 360 and cavity 362 are filled with a number of ribs 364 to increase strength and rigidity of the first arm 330 and the second arm 340. Other variations are contemplated in this disclosure including but not limited to, rectangular, elliptical or combined cross-sectional shapes. Specifically, the arms may have one or more flat sides, one or more rounded sides, or both.

FIG. 10 illustrates another embodiment of syringe handle 400. Syringe handle 400 is similar to the assembly shown in FIGS. 2-5 and common elements have similar reference numerals as the embodiment shown in FIGS. 2-5.

The inner surface 416 of the hub portion further defines a slit 470 to allow for the expansion of the hub portion 410 to accommodate different brands and/or sizes of syringes. Slit 470 is positioned along outer surface 418 of the hub portion and extends through the channel 417 defined by inner surface 416. In some embodiments, the slit 419 is centered on the midway point between the first arm 430 and the second arm 440 on the outer surface of the hub portion 410. In other embodiments, it may be desirable to have the slit 470 located in such a position that slit 470 is biased toward either the first arm 430 or the second arm 440 of the grip portion 420.

The hub portion shown in FIG. 10 may further include a first flange 472 and second flange 474. The first flange 472 extends from and is connected to a side of slit 470 and the second flange 474 extends from and is connected to the opposite side of slit 470. In some embodiments the distance between the first flange 472 and the second flange 474 is fixable. In such embodiments the first flange 472 includes an outer face 476 defining hole 478, and the second flange 474 defines a threaded hole 480. Hole 478 is sized to allow a bolt to pass through such that the threads of said bolt engage threaded hole 480 while the head of said bolt engages outer face 476. In other embodiments the distance between flange 472 and flange 474 may be fixable by another method, including, but not limited to, a clasp, buckle, pin, or clamp.

In other embodiments, a syringe handle may be integrated with a syringe that includes a hub portion and a grip portion. In such embodiments, the hub portion is the body portion of the syringe and includes a barrel portion having an open end configured to receive a plunger. In some embodiments, the syringe may further comprise a necked portion configured to receive a needle and an outside surface. The barrel portion has an elliptical cross section (for example having a generally cylindrical shape) and further defines a central axis along its length. Where the barrel portion is not cylindrical, the barrel portion defines an axis along its length such that said axis passes through the center of gravity of the barrel portion. Each of the first arm and the second arm can be arranged and configured as described above with reference to FIGS. 2-9.

The grip portion includes a first arm and a second arm near the open end of the hub portion. Each of the first arm and the second arm include an end defining a connection point, a distal end, and an outer surface. The first arm is integrally connected to the outer surface of the barrel portion at a connection point near the open end. The second arm is integrally connected to the outer surface of the barrel portion on the outer surface of the barrel portion at a connection point opposite the first arm connection point. The first arm extends from the outer surface of the barrel portion in a radial manner and is biased towards the open end of the hub portion, terminating at a distal end. The second arm extends from the outer surface of the barrel portion in a radial manner and is biased towards the necked end of the body portion, terminating at a distal end. The first and second arms have a central axis extending along the entire length of each arm. The central axis of the first arm, the second arm or both may extend through the hub portion. the central axes of the first arm and the central axis of the second arm of the grip portion may be parallel or coincident. Moreover, the first arm and the second arm of the grip portion may comprise an elliptical cross-section, a rectangular cross section, a rectangular cross section with rounded corners or other similar shapes. The first arm and the second arm may comprise a smooth, continuous and uninterrupted exterior surface. The first arm and the second arm may comprise an elliptical cylinder shape. The arms can be connected at various points on the hub. In one example, at least one arm is connected at a connection point on the hub wherein a cross sectional area of the attachment point is located entirely between the top surface and bottom surface of the hub portion.

The hub can be configured such that either the top or the bottom surface of the hub portion can abut a flange of a syringe when the syringe handle is in an operational position. In particular, the hub can comprise a split cylindrical shape.

The present disclosure further contemplates a wide array of adaptor designs, that may be used to connect the adaptor to devices having an elongate member such as a syringe with one or more projections.

As such, combinations of the exemplary embodiments, particularly with respect to shape, arrangement and properties of the projection(s) is contemplated herein. For instance, in an exemplary embodiment, a syringe adaptor comprises a hub portion comprising an inner surface and an outer surface, and at least one projection extending from the inner surface. A channel defined by the inner surface has a central longitudinal axis. The channel may have essentially any cross section suitable for engaging a syringe barrel. Non-limiting examples include a triangular cross section, an elliptical cross section, a rectangular cross section, rectangular cross section with rounded edges and other similar shapes. A grip portion extends from the hub outer surface. The adaptor is configured to receive a syringe barrel in the channel and at least one projection is configured to engage the barrel.

Of course, the hub portion may have different cross-sectional shapes including elliptical or rectangular cross sections. Thus, the relative shape, position and arrangement of the projection may be described in a variety of ways. For instance, in an exemplary the at least one projection may be described as extending away from an inner surface portion toward an adjacent or opposite inner surface portion. The projection may also be described as extending towards the central longitudinal axis in the channel. In another sense, a projection may be considered to extend radially, circumferentially or both with respect to the inner surface. The projection may be considered to extend radially within the channel.

The hub may comprise two or more projections. The present disclosure contemplates any number of projections as may be beneficial for connecting the adaptor to a particular device, such as a syringe. In one example, the hub comprises a plurality of projections. In particular, at least some of the plurality of projections may be configured to engage a syringe barrel outer surface. The projections may also support each other. Moreover, the projections may be spaced from each other, in contact with each other or both to optimize engagement with the syringe barrel as well as realize any mechanical action for maintaining the engagement. The spacing may be the same between each projection or different. A non-limiting example includes a large array of closely spaced projections, for example resembling a bristle.

Independently of the hub configuration, the projections may have a wide variety of practical shapes. For instance, they may have a cross section that is rectangular, elliptical or both. They may be solid or hollow and have a rounded, flat or pointed distal end. Therefore, shapes such as, hemispherical, pyramidal, cylindrical, conical and combination thereof are contemplated.

In an exemplary embodiment, the hub comprises one or more projections that are short protrusions. In another exemplary embodiments, the projections comprise an elongate member. Accordingly, the diameter of a protrusion may be the same or vary along its entire length. The projection may be curved, straight or both. In an exemplary embodiment, the curvature of at least one projection is configured to slidingly receive at least a portion of a syringe barrel. The projection degree of curvature may be less than, greater than or the same as the curvature of the inner surface of the hub. Moreover, the projection may have different degrees of flexibility. Moreover, the projection may be biased in one or more directions including, but not limited to, towards or away from another inner surface portion, or towards or away from the nearest inner surface portion. Stated differently, the projection may be configured to deflect away from the central longitudinal axis or an inner surface portion. In a particular example, a projection may be supported by a surface portion, another projection or both to optimize the degree of deflection. Advantageously, such designs may enhance engagement with the barrel.

In an exemplary embodiment, the projection comprises a foot portion. The foot portion may be located at the distal end of the projection and assume essentially any shape suitable for the intended application. For instance, the foot portion may have surfaces that are concave, convex, flat, sharp, or a combination thereof. Such surfaces may enhance engagement with various syringe barrel shapes and sizes.

In an exemplary embodiment the foot portion is rectangular. The projection itself may have a longitudinal axis, whereby the foot portion is angled or orthogonal thereto.

As provided earlier, the inner surface portion may support or engage with a projection. For example, the inner surface may be configured to mate with at least a portion of a projection when the projection is deflected towards said inner surface portion. In an exemplary embodiment, the inner surface comprises at least one recess, such as but not limited to a recess, configured to engage a projection. The recess may have any shape permitting it to engage with any portion of the projection, such as the foot portion. In an exemplary embodiment at least one surface of the projection (with or without a foot) mates with at least one surface of the recess. In another exemplary embodiment the recess releasably couples with the projection.

With respect to manufacturing, the projections may be integrally formed with the hub portion, formed separately or both. Thus, at least one projection may comprise the same material as the hub. The projections may comprise different material from each other. The choice of material may depend on any requirements in the industry such as health safety as well as mechanical and practical considerations. In an exemplary embodiment, the adaptor is modular permitting addition of one or more projections. For instance, one or more projections of the same or different sizes can be attached to the hub portion. This may permit accommodating different syringe sizes. A non-limiting example of the projection may be a U-shaped object that is slid onto the hoop. It may slide onto a recess (e.g., linear groove) that partially or completely extends the axial length of the hub. It may also slide onto an unmodified inner surface. The add-on projection may be one piece (e.g., interconnected projections) such that the user may chose the projection size based on the syringe and simply engage (interlock, mate, etc.) the two pieces (hub and projection(s) unit) together. In still a further embodiment, the projection and one or more arms may be connected such that engaging both pieces adds arms and projections at the same time. Thus, the user has even more options for the size/shape of projections and arms.

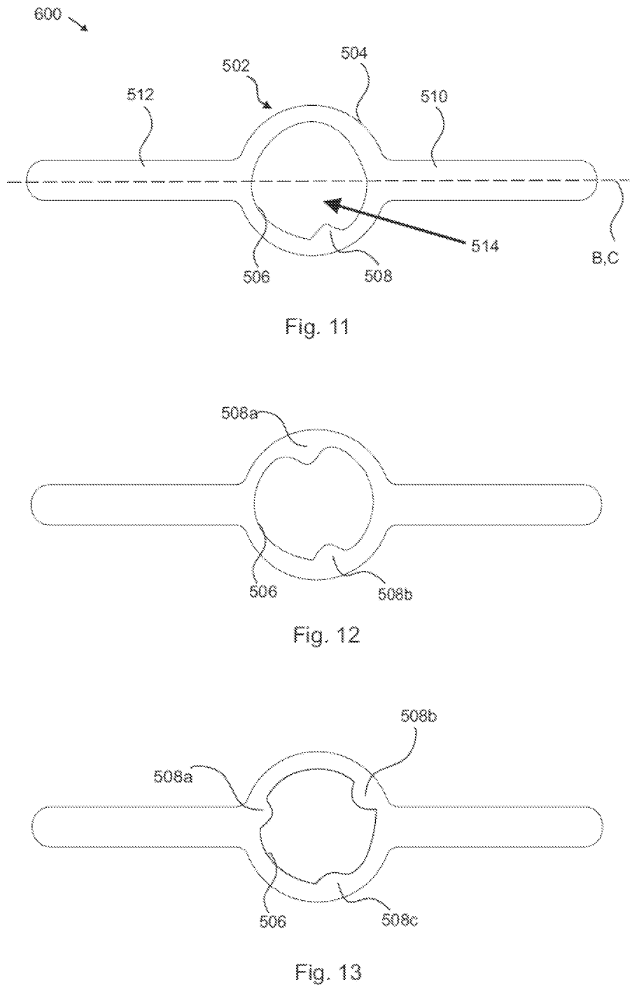

Embodiments of the present disclosure are further exemplified in FIGS. 11-45. In the exemplary embodiment depicted in FIG. 11 the syringe adaptor 500 comprises a hub 502 having an outer surface 504 and an inner surface 506. A first arm 510 and a second arm 512 extend from the outer surface 504 of the hub portion 502. Axis B is a central longitudinal axis extending through the first arm 510 and Axis C is a central longitudinal axis extending through the second arm 512. A channel 514 is defined by the inner surface 506 of the hub 502, and is configured to receive the barrel of a syringe. The hub portion further comprises a projection 508 extending from the inner surface 506, which is configured to engage a syringe barrel placed in the channel 514. In FIG. 12 the hub portion comprises a first projection 508a and a second projection 508b extending from the inner surface 506. The projections 508a and 508b are shown as located generally opposite each other though their relevant position can vary. In an exemplary embodiment, the projections 508a and 508b are not directly across from each other. That is, they are offset such that a line connecting 508a and 508b does not pass through the center of the hub 502. Stated differently, the two projections are not located at noon and 6 o'clock. In a specific example, the two projections are relatively positioned at about 1 and about 5 o'clock. In FIG. 13, a first projection 508a, a second projection 508b and a third projection 508c are shown where each projection is generally equally spaced from an adjacent projection. Again, the spacing may vary here as well. FIGS. 11-13 further exemplify pointed and rounded projections.

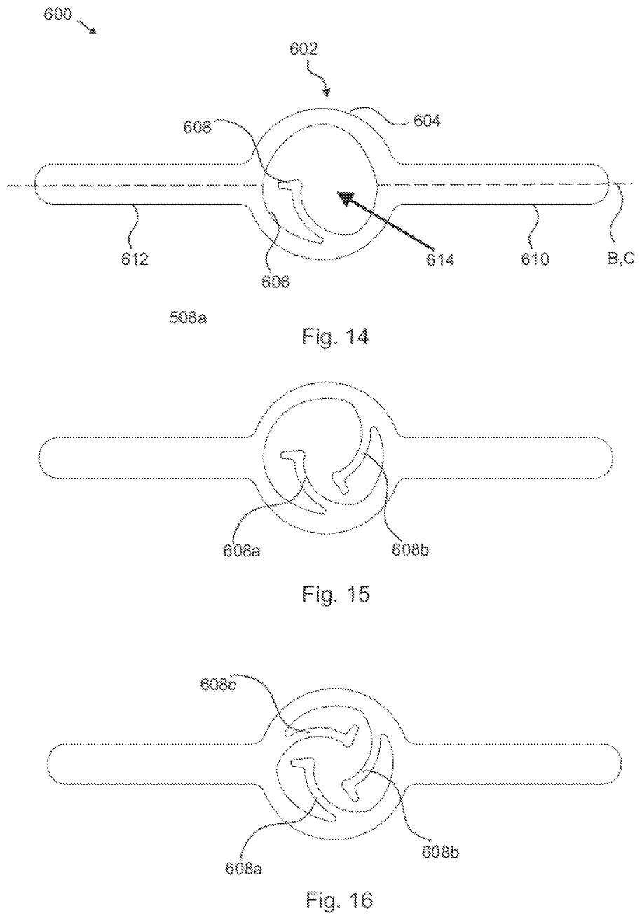

FIGS. 14-16 depict several embodiments with respect to projection, shapes, sizes and arrangements. For instance, in FIG. 14, the adaptor 600 comprises a hub portion 602 having an outer surface 604 and an inner surface 606. A first arm 610 and a second arm 612 extend from the outer surface 604 of the hub portion 602. Axis B is a central longitudinal axis extending through the first arm 610 and Axis C is a central longitudinal axis extending through the second arm 612. A channel 614 is defined by the inner surface 606 of the hub 602, and is configured to receive the barrel of a syringe.

Here, the projection 608 is generally a curved elongate member with a modified distal end. One or more such projections may be configured to engage with a syringe barrel. FIG. 15 depicts a variation with two projections 608a and 608b, while FIG. 16 depicts three projections 608a, 608b and 608c. The projections are generally shown as identical, but they may be different in shape, size or location as required for the application.

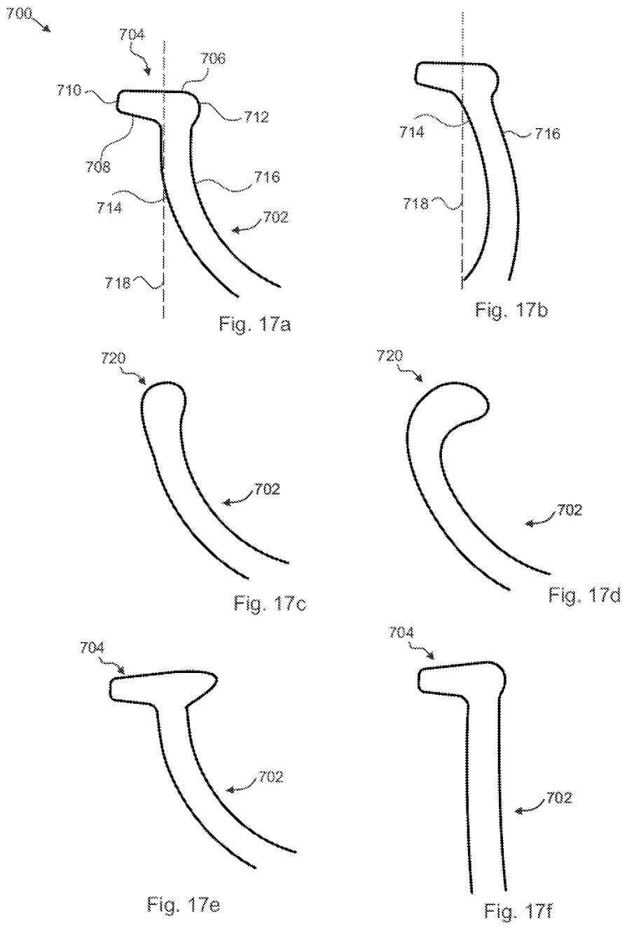

FIGS. 17a-f depict cross-sectional views for several non-limiting variations of projections. Starting with FIG. 17a, the projection 700 comprises an elongate body portion 702 and a foot portion 704 at the distal end of the body portion 702. The foot portion has a top side 706 and an opposing a bottom side 708, as well as a front side 710 and an opposing back side 712. Here, the body portion 702 is curved and comprises a first side 714 and second side 716. The curvature shown is with respect to a longitudinal axis 718 generally through the center of the foot portion 704. As shown, the first side 714 and second side 716 are concave with respect to the axis 718. In contrast, the body portion 702 of the projection in FIG. 17b is concave with respect to the axis 718.

FIGS. 17c and 17d illustrate variations where the projection 700 does not comprise a foot portion whereby the distal portion 720 terminates in in various regular or irregular shapes. Essentially, all shapes are contemplated herein. For example, the distal end may be flat, rounded (e.g., a knob), serrated, pitted, pointed, U-shape cross-sectioned, V-shape cross sectioned, textured (e.g., to improve traction), smooth, faceted, or any combination thereof. FIG. 17 depicts a variation where the foot portion is “hammer-shaped” and in FIG. 17f, the body portion 702 is substantially straight.

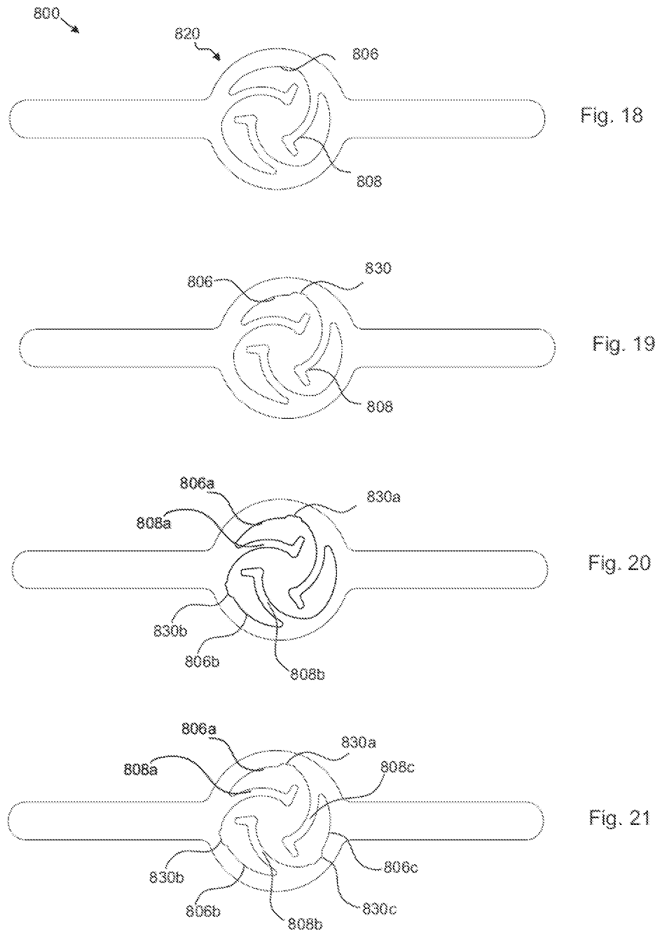

FIGS. 18-21 illustrate adaptors 800 with various inner surface 806 recess 830 configurations. For instance, in FIG. 18, the inner surface 806 of the hub is relatively smooth and uninterrupted. In FIG. 19, the inner surface 806 comprises one recess 830. In FIG. 20 an inner surface portion 806a comprises a recess 830a which may receive, engage or otherwise come into contact with an adjacent projection 808a. Similarly, inner surface portion 806b has a recess 830b and a complimentary projection 808b. FIG. 21 shows three such combinations of recesses and projections.

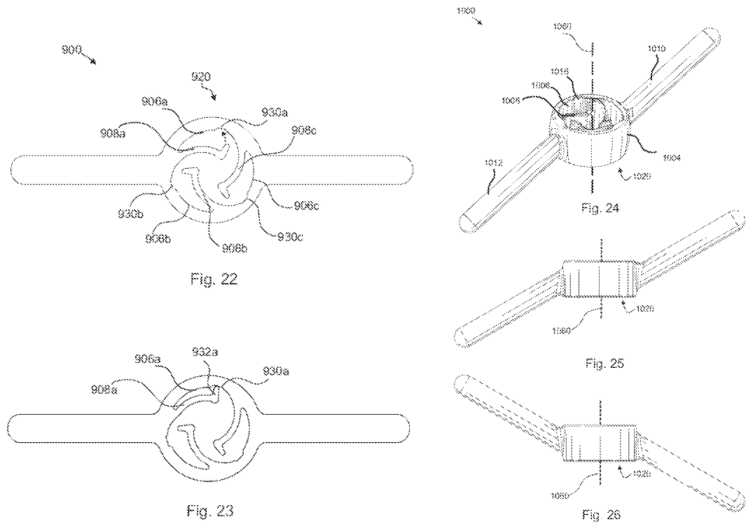

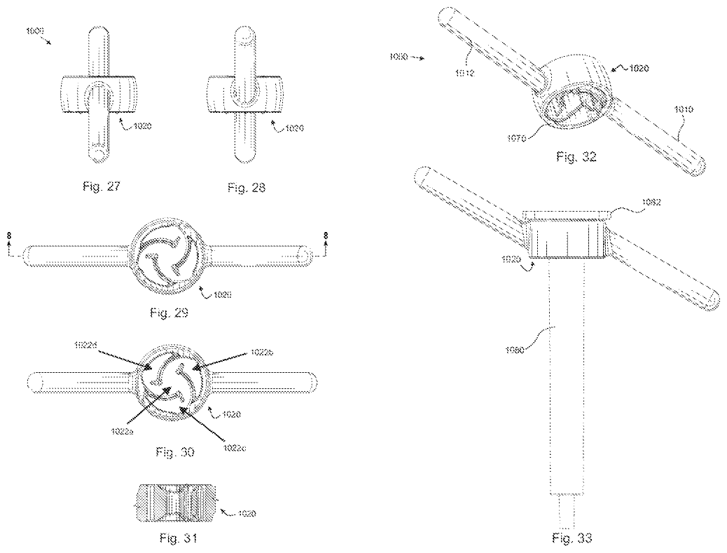

As mentioned earlier, the projection may deflect toward an inner surface portion which may or may not include a recess. In FIG. 22, the adaptor 900 comprises a hub portion which includes three projection and recess pairs. As shown, projection 908a deflects towards the recess 930a on the inner surface 906a. FIG. 23 shows the complete deflection of the projection 908a whereby it contacts the recess 930. Although not shown, the other projections 908b and 908c may also deflect towards inner surface portions 906b and 906c. In other words, upon engaging a syringe barrel, one, two or three of the projections may deflect towards the inner surface. The distance and degree of deflection may be the same for two or all three of the projections. Alternatively, the deflection may be different for each. For instance, some may fully deflect to contact the inner surface, some may partially deflect, and some may not deflect at all. This of course can depend in part on the size of the syringe and the manner of placement of the adaptor on a syringe barrel, among other factors. In an exemplary embodiment, the projection may be configured to deflect towards a protrusion extending from the inner surface. Specifically, the projection may releasably engage the protrusion.

FIGS. 24-33 provide further views of an exemplary embodiment. For instance, in FIG. 24, the adaptor 1000 comprises a hub 1020 having an outer surface 1004 and an inner surface 1006. A first arm 1010 and a second arm 1012 which extend from the outer surface 1004 of the hub portion 1020. The adaptor comprises a central longitudinal axis 1060 extending through the hub. A channel is defined by the inner surface 1006 of the hub 1020, and is configured to receive the barrel of a syringe. The hub portion further comprises several projections 1008 extending from the inner surface 1006, which is configured to engage a syringe barrel placed in the channel FIGS. 25-28 provide different side views of the syringe in FIG. 24. In particular, FIG. 31 provides a partial cross-sectional view of FIG. 29, along lines 8-8.

In one sense the channel which defined by the inner surface of the hub 1006 may be further defined or subdivided by the projections. For instance, as shown in FIG. 30, there may be a channels 1022a, 1022b, 1022c and 1022d. Accordingly, there a syringe will likely be inserted into the channel 1022a. However, based on the design of the adaptor, including the number, shape and size of the projections, the syringe may be received in other channels.

FIG. 32 shows a bottom perspective view of an adaptor 100, having a hub portion 1020 a first arm 1010 and a second arm 1012, as well as a bottom surface 1070 of the hub 1020.

In practice, the adaptor 100 is typically slid onto the syringe barrel 1080 as shown in FIG. 33 where the top surface or bottom surface 1070 may contact the syringe flange 1082.

FIGS. 33-47 illustrate an adaptor design having a slit. In these exemplary embodiments, the adaptor 1100 comprises a hub 1120 having an inner surface 1106 and an outer surface 1104. The hub portion 1120 has a top side 1196 and a bottom side 1198 which may abut the syringe flange when in an operational position, thereby advantageously allowing for use on either side. A first arm 1110 and a second arm 1112 extend from the outer surface 1104. The inner surface comprises three projections 1106. In FIG. 35, projections 1108a, 1108b and 1108c are shown as generally equally distanced from the adjacent projection. Of course, the present disclosure contemplates any number of projections, including designs without any projections as well as various placement and spacing between the one or more projections.

As with other exemplary embodiments, the inner surface 1106 defines a channel 1122 for receiving a syringe barrel. In the exemplary embodiments a slit 1190 is shown interrupting the continuity of the hub portion. Advantageously the slit permits deflection of the hub wall when receiving a syringe barrel to accommodate for different widths. Thus, in a sense, the slit 1120 permits expansion of the channel 1122.

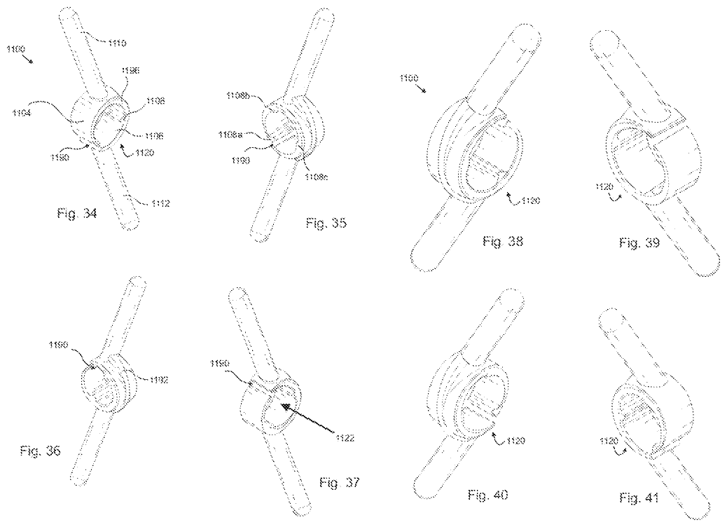

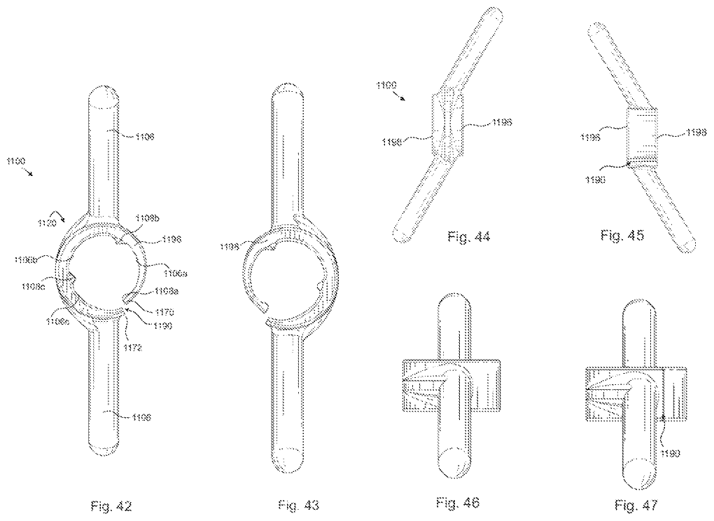

FIG. 42 provides further details, where the adaptor 1110 comprises a hub portion 1120 having an inner surface subdivided into inner surfaces 1106a, 1106b and 1106c. The hub has a slit 1190 defined by the first end 1170 and second end 1172 of the hub. As shown, the distal ends 1170 and 1190 are offset. However, the present disclosure contemplates various slit shapes and sizes as well as non-offset and mating distal ends. Also shown is a first projection 1108a located near the first end 1170, a second projection 1108b located between the inner surfaces 1106a and 1106b, as well as third projection 1108c located between inner surfaces 1106b and 1106c. FIGS. 44-47 present additional views of this embodiment. As provided earlier, the inner surface of the syringe adaptor may or may not have one or more recesses.

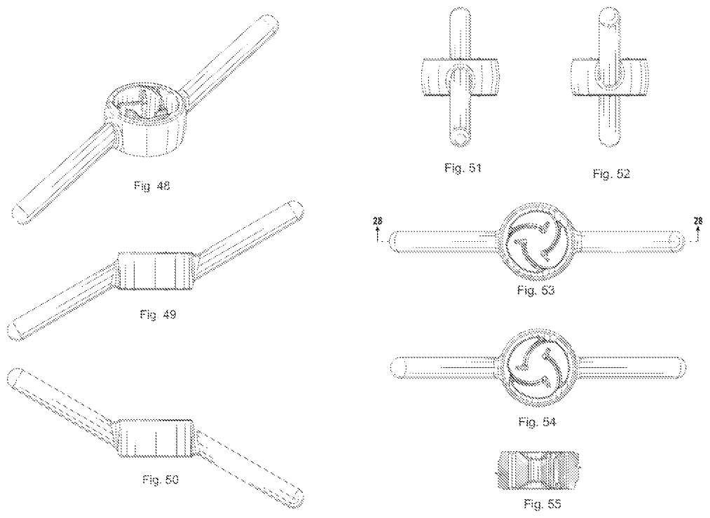

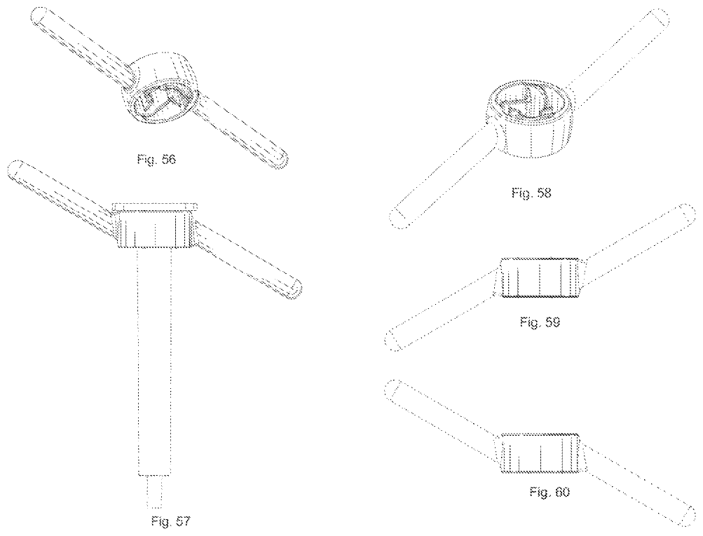

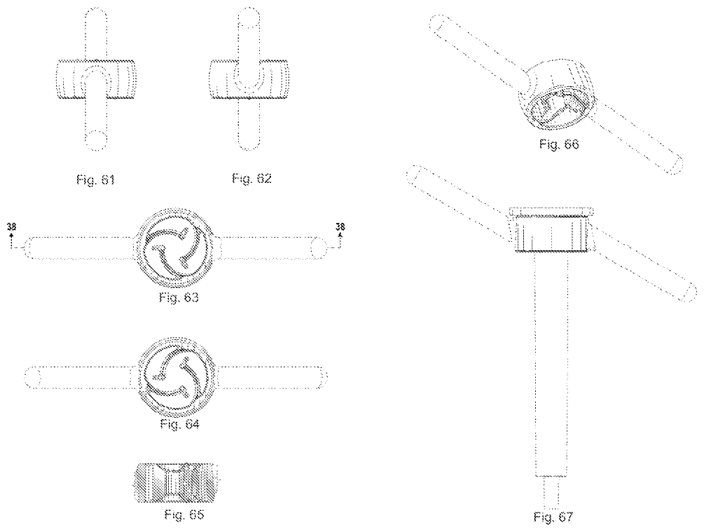

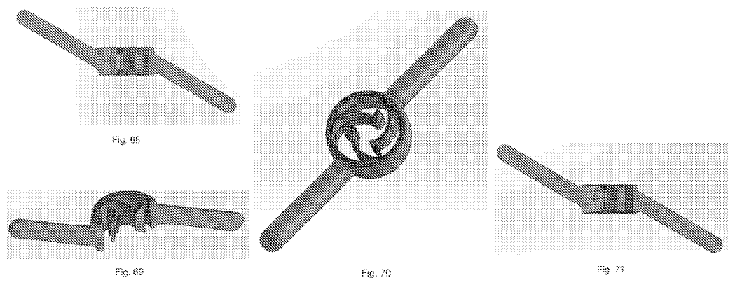

As provided earlier, the inner surface of the syringe adaptor may or may not have one or more recesses. Embodiments without an inner surface recess are further exemplified in FIGS. 48-72.

It will be clear that the systems and methods described herein are well adapted to attain the ends and advantages mentioned as well as those inherent therein. Those skilled in the art will recognize that the systems within this specification may be implemented in many manners and as such is not to be limited by the foregoing exemplified embodiments and examples. In this regard, any number of the features of the different embodiments described herein may be combined into one single embodiment and alternate embodiments having fewer than or more than all of the features herein described are possible.

While various embodiments have been described for purposes of this disclosure, various changes and modifications may be made which are well within the scope contemplated by the present disclosure. Numerous other changes may be made which will readily suggest themselves to those skilled in the art and which are encompassed in the spirit of the disclosure.

What is claimed is:

1. A syringe adaptor comprising:
a grip portion;
a hub portion comprising an inner surface and an outer surface;
a channel defined by the inner surface; and
a central axis passing through the channel; and
at least one projection extending away from the inner surface,
wherein the at least one projection is configured to engage a syringe barrel received in the channel, and
wherein the grip portion extends from the hub outer surface away from the central axis.

2. The syringe adaptor according to any preceding claim, wherein the at least one projection generally extends towards the central axis.

3. The syringe adaptor according to any preceding claim, wherein the at least one projection extends away from an inner surface portion toward an adjacent portion of the surface.

4. The syringe adaptor according to any preceding claim comprising two or more projections spaced from each other.

5. The syringe adaptor according to any preceding claim comprising three projections equally spaced from each other.

6. The syringe adaptor according to any preceding claim wherein the at least one projection comprises a rectangular cross section with rounded edges.

7. The syringe adaptor according to any preceding claim comprising at least two connected projections.

8. The syringe adaptor according to any preceding claim wherein at least one projection is an elongate member.

9. The syringe adaptor according to any preceding claim wherein at least one projection comprises a varying diameter along its length.

10. The syringe adaptor according to any preceding claim wherein the at least one projection comprises the same diameter along its entire length.

11. The syringe adaptor according to any preceding claim wherein the curvature of the at least one projection is configured to slidingly receive at least a portion of a syringe barrel outer surface.

12. The syringe adaptor according to any preceding claim wherein the at least one projection has a degree of curvature that is less than or greater than a curvature of the inner surface.

13. The syringe adaptor according to any preceding claim wherein the at least one projection is biased way from the nearest inner surface portion.

14. The syringe adaptor according to any preceding claim wherein the at least one projection comprises a foot portion.

15. The syringe adaptor according to any preceding claim wherein the inner surface comprises at least one recess configured to engage a projection.

16. The syringe adaptor according to any preceding claim wherein the inner surface comprises at least one recess configured to mate with the foot portion of a projection.

17. The syringe adaptor according to any preceding claim wherein the inner surface comprises at least one recess configured to releasably couple with the foot portion of a projection.

18. The syringe adaptor according to any preceding claim, further comprising a projection configured to deflect towards and mate with a protrusion extending from the inner surface.

19. A syringe adaptor comprising:
a hub portion comprising an outer surface, an inner surface, a top surface and a bottom surface, the inner surface being curved and configured to receive a portion of a syringe body;
and each of the top surface and the bottom surface being adjacent to both the outer surface and the inner surface;
a grip portion; and
at least one projection extending from the inner surface.

20. A method of manufacturing a syringe adaptor comprising:
forming a hub portion comprising an inner surface and an outer surface, such that a channel is defined by the inner surface, said channel having a central longitudinal axis;
forming a grip portion which extends from the outer surface of the hub portion;
forming at least one projection extending from the inner surface of the hub portion wherein the at least one projection is configured to engage a syringe barrel.
